SEQUENCE LISTING (3 sequences)

SEQ ID NO: 1 - RNA, 19 nt, Homo sapiens
gcauuuccagcucguuuga

SEQ ID NO: 2 - RNA, 19 nt, Mus musculus
cgagugagagaugagccug

SEQ ID NO: 3 - protein, 13 aa, Artificial Sequence
  synthetic construct
RPDMAPELTPEEE

(19) United States
(12) Patent Application Publication
(10) Pub. No.: US 2009/0156523 A1
(43) Pub. Date: Jun. 18, 2009

(54) METHODS AND COMPOSITIONS FOR MODULATING FOXO1 ACTIVITY AND INSULIN SIGNALING

(75) Inventors: Charles Cho, San Diego, CA (US); Sumit Chanda, La Jolla, CA (US); Peter G. Schultz, La Jolla, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 11/995,526

(22) PCT Filed: Jul. 11, 2006
(86) PCT No.: PCT/US2006/027047

§ 371 Date: Jan. 11, 2008

Related U.S. Application Data
(60) Provisional application No. 60/698,332, filed on Jul. 11, 2005.

(51) Int. Cl. A61K 31/7088; C12Q 1/00

(52) U.S. Cl. 514/44; 435/4

(57) ABSTRACT

The invention provides methods for modulating insulin signaling pathway and methods for treating insulin resistance. The methods employ agents which modulate FOXO1 subcellular localization. The invention also provides methods of screening for compounds that modulate insulin signaling related activities such as gluconeogenesis and cell proliferation.

### CROSS-REFERENCE TO RELATED APPLICATIONS

This patent application claims the benefit of priority under 35 U.S.C. §119(e) to U.S. Provisional Patent Application No. 60/698,322, filed Jul. 11, 2005. The disclosure of the priority application is incorporated herein by reference in its entirety and for all purposes.

### FIELD OF THE INVENTION

The present invention relates to methods of modulating insulin signaling and methods of identifying novel compounds that regulate gluconeogenesis and insulin signaling.

### BACKGROUND OF THE INVENTION

FOXO1 (also termed FKHR) is a member of the FOXO subfamily of Forkhead transcription factors which are important targets for insulin and growth factor signaling. The FOXO family of transcription factors has been implicated in diverse cellular processes including gluconeogenesis, differentiation, cell proliferation, and stress responses. For example, they mediate effects of insulin and growth factors on gene expression downstream from phosphatidylinositol 3-kinase (PI3K) and protein kinase B (PKB; also known as Akt). In the absence of insulin stimulation, FOXO1 is present in the nucleus of hepatocytes where it participates in the transcription of genes such as glucose-6-phosphatase, which catalyzes the terminal step of gluconeogenesis. Upon insulin stimulation, FOXO1 is excluded from the nucleus as a consequence of an Akt-mediated phosphorylation. This results in suppression of gluconeogenesis. In other cell types, FOXO proteins also stimulate the expression of proteins that inhibit cell cycle progression and protein that promote cells death.

The ability to suppress transactivation by FOXO Forkhead proteins is important for insulin to regulate hepatic production of IGFBP-1 and glucose and for effects of growth factors on cell proliferation and survival. Dysregulation of FOXO1 could contribute to insulin resistance in non-insulin dependent diabetes. See, Martin et al., J Mol Endocrinol. 29: 205-22, 2002; Streeper et al., J Biol. Chem. 276:19111-8, 2001; Ayala et al., Diabetes. 48: 1885-9, 1999; and Seoane et al., J Biol Chem. 272(43): 26972-7, 1997.

Compounds that modulate FOXO1 subcellular localization provide means of regulating gluconeogenesis and potential treatment of insulin resistance. Molecules that regulate FOXO1 subcellular localization also provide targets for medicinal intervention of the insulin signaling pathway. There is a need in the art for better means for modulating insulin signaling related activities such as gluconeogenesis, and for treating diseases and conditions caused by or associated with aberrant insulin signaling and abnormal gluconeogenesis. The present invention addresses this and other needs.

### SUMMARY OF THE INVENTION

In one aspect, the invention provides methods of treating or ameliorating insulin resistance in a subject. The methods entail administering to the subject a pharmaceutical composition comprising an effective amount of a compound which down-regulates cellular level or enzymatic activity of PTP-MEG2. Some of the methods employ an agent which down-regulates expression level of PTP-MEG2. For example, the agent used in these methods can be short interfering RNAs (siRNAs) or short hairpin RNA (shRNAs) that specifically target PTP-MEG2. Other suitable agents for practicing these methods include microRNAs, anti-sense nucleic acids, and complementary DNAs.

In another aspect, the invention provides methods for identifying compounds that modulate insulin signaling-related activities. The methods involve first screening test compounds to identify one or more modulating compounds which modulate an FOXO1 localization regulator disclosed herein. The identified modulating compounds are then tested for ability to modulate an insulin signaling related activity. In some of the methods, the modulating compounds down-regulate the FOXO1 localization regulator. In some other methods, the modulating compounds up-regulate the FOXO1 localization regulator. In some methods, the FOXO1 localization regulator employed in the screening is an enzyme, and the modulating compounds modulate an enzymatic activity of the FOXO1 localization regulator. In some methods, the modulating compounds modulate expression of the FOXO1 localization regulator. The FOXO1 localization regulator employed in the methods can be an inhibitor of FOXO1 nuclear localization or a stimulator of FOXO1 nuclear localization shown in Table 1.

In some embodiments, the FOXO1 localization regulator employed in the screening methods is PTP-MEG2. In some of these methods, the modulating compounds inhibit the phosphatase activity of PTP-MEG2. In some other methods, the modulating compounds down-regulate cellular level of PTP-MEG2.

In some of the screening methods, the identified modulating compounds are tested for ability to modulate subcellular localization of FOXO1. For example, the compounds can be tested for ability to inhibit FOXO1 nuclear localization. In some other methods, the identified modulating compounds are tested for ability to modulate FOXO1-mediated expression of an insulin signaling pathway member, e.g., glucose-6-phosphatase. Some of these methods are directed to identifying compounds that down-regulate expression of the insulin signaling pathway member.

In a related aspect, the invention provides methods for identifying an agent that modulates nuclear localization of transcription factor FOXO1. These methods entail screening test compounds to identify one or more modulating compounds which modulate an FOXO1 localization regulator disclosed herein. The identified modulating compounds are then tested for ability to modulate FOXO1 nuclear localization. In some of these methods, the FOXO1 localization regulator employed is a kinase, and the test compounds are screened for ability to modulate its kinase activity. In some other methods, the employed FOXO1 localization regulator is a phosphatase, and the test compounds are examined for activity in modulating its phosphatase activity. In some embodiments, the employed phosphatase is PTP-MEG2. In these methods, the identified modulating compounds can either up-regulate or down-regulate the expression or another biological activity of the FOXO1 localization regulator. In some of the methods, the identified modulating compounds are screened for ability to stimulate or inhibit FOXO1 nuclear localization. For example, the compounds can be examined for activity in modulating subcellular localization of FOXO1 in cells that express a FOXO1 protein. In some embodiments, modulation of FOXO1 nuclear localization is assessed with cells (e.g., U2OS cells) that express a GFP-FOXO1 fusion protein.

In another aspect, the invention provides methods for identifying an agent that inhibits tumorigenesis. These methods entail screening test compounds to identify one or more modulating compounds which modulate an FOXO1 localization regulator described herein, and then testing the identified compounds for ability to inhibit tumorigenesis. The methods can also include examining the modulating compounds for ability to stimulate FOXO1 nuclear localization prior to testing for their antitumor activity. Typically, the modulating compounds identified in the screening methods up-regulates expression or another biological activity of the FOXO1 localization regulator. In some of the methods, the identified modulating compounds are tested for ability to inhibit proliferation of a tumor cell in vitro.

A further understanding of the nature and advantages of the present invention may be realized by reference to the remaining portions of the specification and claims.

### DETAILED DESCRIPTION

### I. Overview

The invention is predicated in part on the discoveries by the present inventors of genes that regulate the subcellular localization of FOXO1. FOXO1 is a transcription factor that regulates the expression of several genes including glucose-6-phosphatase, Fas ligand, and p27. FOXO1 activity is negatively regulated by the kinase Akt, a downstream target of insulin and other growth factors. As detailed in the Examples below, the present inventors examined the impact of approximately 4,600 full-length human and mouse cDNAs on the subcellular localization of FOXO1. Specifically, the cDNAs were individually transfected into the U2OS cells along with a reporter construct which expressed a GFP-FOXO1 fusion protein. Subcellular localization of FOXO1 in the cells was examined by high throughput fluorescence microscopy imaging.

Average FLIN (fractional fluorescence in the nucleus) values were determined for each cDNA in the collection. cDNAs which induced a change in nuclear localization of more than 3.5 standard deviations in FLIN values were selected for further reconfirmation. Among the confirmed screen hits, some cDNAs were found to induce FOXO1 nuclear localization, while others led to FOXO1 sequestration in the cytoplasm. These cDNAs and their encoded polypeptides are termed herein “regulators of FOXO1 subcellular localization” or “FOXO1 localization regulators” They encompass both “stimulators of FOXO1 nuclear localization” which induce FOXO1 nuclear localization by >3.5 σ (shown in Table 1) and “inhibitors of FOXO1 nuclear localization” which decrease FOXO1 nuclear localization by <−2 σ (shown in Table 2).

TABLE 1
Stimulators of FOXO1 Nuclear Localization

| | Accession | | | | SD from |
|---|---|---|---|---|---|
| | No. | Symbol | Description | Reference | mean |
| 1 | BC006796 | Pik3r2 | phosphatidylinositol 3- | Ueki et al Proc Natl | 9.6σ |
| | | | kinase, regulatory subunit, | Acad Sci USA. | |
| | | | polypeptide 2 (p85 beta) | 99(1): 419-24 (2002) | |
| 2 | BC011118 | Cebpa | CCAAT/enhancer binding | Christy et al Proc. | 9.4σ |
| | | | protein (C/EBP), alpha | Natl. Acad. Sci. | |
| | | | | U.S.A. 88 (6), 2593-2597 | |
| | | | | (1991) | |
| 3 | BC010863 | PTPN9 (PTP- | protein tyrosine | Gu et al Proc Natl | 9.0σ |
| | | MEG2) | phosphatase, non-receptor | Acad Sci USA. | |
| | | | type 9 | 89(7): 2980-4 (1992) | |
| 4 | BC008512 | PTPN18 | protein tyrosine | Huang et al | 8.1σ |
| | | | phosphatase, non-receptor | Oncogene 13 (7), | |
| | | | type 18 | 1567-1573 (1996) | |
| 5 | BC011286 | 3100004P22Rik | RIKEN cDNA | Kawai et al Nature | 6.3σ |
| | | | 3100004P22 | 409 (6821), 685-690 | |
| | | | | (2001) | |
| 6 | BC018480 | Guca1b | guanylate cyclase activator | Mendez et al Proc. | 6.2σ |
| | | | 1B | Natl. Acad. Sci. | |
| | | | | U.S.A. 98 (17), | |
| | | | | 9948-9953 (2001) | |
| 7 | BC015180 | HOXA3 | homeo box A3 | McAlpine and | 6.0σ |
| | | | | Shows Genomics 7 | |
| | | | | (3), 460 (1990) | |
| 8 | BC010315 | Tdg | thymine DNA glycosylase | De Gregorio et al | 5.9σ |
| | | | | Mamm. Genome 7 | |
| | | | | (12), 909-910 | |
| | | | | (1996) | |
| 9 | BC006704 | Dyrk3 | dual-specificity tyrosine- | Becker et al J. Biol. | 4.9σ |
| | | | (Y)-phosphorylation | Chem. 273 (40), | |
| | | | regulated kinase 3 | 25893-25902 (1998) | |
| 10 | BC011211 | Mat1a | methionine | Sakata et al J. Biol. | 4.9σ |
| | | | adenosyltransferase I, | Chem. 268 (19), | |
| | | | alpha | 13978-13986 (1993) | |
| 11 | BC016616 | 5830417I10Rik | RIKEN cDNA 5830417I10 | Strausberg et al | 4.5σ |
| | | | gene | Proc. Natl. Acad. | |
| | | | | Sci. U.S.A. 99 (26), | |
| | | | | 16899-16903 (2002) | |
| 12 | BC013639 | Hoxb13 | homeo box B13 | Zeltser et al | 4.4σ |
| | | | | Development 122 | |
| | | | | (8), 2475-2484 | |
| | | | | (1996) | |
| 13 | BC015212 | ZNF291 | zinc finger protein 291 | Nagase et al DNA | 4.1σ |
| | | | | Res. 7 (2), 143-150 | |
| | | | | (2000) | |
| 14 | BC004793 | Pcbp1 | poly(rC) binding protein 1 | Makeyev et al J. | 4.0σ |
| | | | | Biol. Chem. 274 | |
| | | | | (35), 24849-24857 | |
| | | | | (1999) | |
| 15 | BC008602 | PSTPIP1 | proline-serine-threonine | Li et al EMBO J. 17 | 3.8σ |
| | | | phosphatase interacting | (24), 7320-7336 | |
| | | | protein 1 | (1998) | |
| 16 | BC016509 | SCAMP4 | secretory carrier membrane | Fernandez-Chacon | 3.6σ |
| | | | protein 4 | and Sudhof J. | |
| | | | | Neurosci. 20 (21), | |
| | | | | 7941-7950 (2000) | |

TABLE 2
Inhibitors of FOXO1 Nuclear Localization

| | Accession | | | | SD from |
|---|---|---|---|---|---|
| | No. | Symbol | Description | Reference | mean |
| 1 | BC001263 | SGK | serum/glucocorticoid | Waldegger et al | −2.0σ |
| | | | regulated kinase | Proc. Natl. Acad. | |
| | | | | Sci. U.S.A. 94 (9), | |
| | | | | 4440-4445 (1997) | |
| 2 | BC003739 | Fkbp8 | FK506 binding protein 8 | Pedersen et al | −2.3σ |
| | | | | Electrophoresis 20 | |
| | | | | (2), 249-255 (1999) | |
| 3 | BC011198 | Apoa5 | apolipoprotein A-V | Pennacchio et al | −2.5σ |
| | | | | Science 294 (5540), | |
| | | | | 169-173 (2001) | |
| 4 | BC000995 | SFN | stratifin | Leffers et al J. Mol. | −2.6σ |
| | | | | Biol. 231 (4), 982-998 | |
| | | | | (1993) | |
| 5 | BC012035 | TLOC1 | translocation protein 1 | Daimon et al | −3.0σ |
| | | | | Biochem. Biophys. | |
| | | | | Res. Commun. 230 | |
| | | | | (1), 100-104 (1997) | |
| 6 | BC010735 | EEF1A1 | eukaryotic translation | Brands et al Eur. J. | −3.0σ |
| | | | elongation factor 1 alpha 1 | Biochem. 155 (1), | |
| | | | | 167-171 (1986) | |
| 7 | BC016618 | LCP2 | lymphocyte cytosolic | Jackman et al J. | −3.2σ |
| | | | protein 2 | Biol. Chem. 270 | |
| | | | | (13), 7029-7032 | |
| | | | | (1995) | |
| 8 | BC011153 | Sqrdl | sulfide quinone reductase- | Mootha et al Cell | −3.4σ |
| | | | like (yeast) | 115 (5), 629-640 | |
| | | | | (2003) | |
| 9 | BC015326 | SGKL | serum/glucocorticoid | Kobayashi et al | −3.5σ |
| | | | regulated kinase-like | Biochem. J. 344 PT | |
| | | | | 1, 189-197 (1999) | |
| 10 | BC003623 | YWHAZ | tyrosine 3- | Zupan et al J. Biol. | −3.9σ |
| | | | monooxygenase/tryptophan | Chem. 267 (13), | |
| | | | 5-monooxygenase | 8707-8710 (1992) | |
| | | | activation protein, zeta | | |
| | | | polypeptide | | |
| 11 | BC020963 | YWHAG | tyrosine 3- | Autieri et al Cell | −4.1σ |
| | | | monooxygenase/tryptophan | Growth Differ. 7 | |
| | | | 5-monooxygenase | (11), 1453-1460 | |
| | | | activation protein, gamma | (1996) | |
| | | | polypeptide | | |

One of the identified regulators of FOXO1 subcellular localization, PTP-MEG2, was subject to further analysis. PTP-MEG2 is a lipid-binding non-receptor protein tyrosine phosphatase. It was found that PTP-MEG2 negatively regulates insulin receptor activation and strongly induces FOXO1 nuclear localization in an activity-dependent manner. In addition, it was observed that overexpression of PTP-MEG2 decreases the phosphorylation of insulin receptor in cultured cells, and RNAi-mediated reduction of PTP-MEG2 expression in HepG2 cells conversely potentiates insulin receptor phosphorylation. Further, it was fund that PTP-MEG2 expression levels are elevated in liver under fasting conditions in the mouse. Finally, it was observed that treating diabetic mice with agents which deplete hepatic expression of PTP-MEG2 reduces insulin resistance and improves insulin sensitivity.

In accordance with these discoveries, the present invention provides methods for identifying compounds that modulate insulin signaling pathway in general and FOXO1 activity in particular. Employing compounds (e.g., siRNAs, shRNAs or small molecule organic compounds) which modulate the FOXO1 localization regulators, the invention further provides methods for regulate insulin signaling in various therapeutic applications. For example, compounds which down-regulate PTP-MEG2 cellular level or enzymatic activity can be employed for and treating insulin resistance in human or non-human subjects. The following sections provide guidance for making and using the compositions of the invention, and for carrying out the methods of the invention.

### II. DEFINITIONS

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more molecules (e.g., a test agent and a polypeptide) or combining molecules and cells (e.g., a test agent and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

A “heterologous sequence” or a “heterologous nucleic acid,” as used herein, is one that originates from a source foreign to the particular host cell, or, if from the same source, is modified from its original form. Thus, a heterologous gene in a host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology.

A “host cell,” as used herein, refers to a prokaryotic or eukaryotic cell to which a heterologous polynucleotide can be introduced. The polynucleotide can be introduced into the cell by any means, e.g., electroporation, calcium phosphate precipitation, microinjection, transformation, viral infection, and/or the like.

As used herein, the term “FOXO1 localization regulator” or “modulator of FOXO1 subcellular localization” refers to both stimulators of FOXO1 nuclear localization shown in Table 1 and inhibitors of FOXO1 nuclear localization shown in Table 2. The term encompasses the genes shown in the tables and also polypeptides encoded by these genes.

The term “sequence identity” in the context of two nucleic acid sequences or amino acid sequences refers to the residues in the two sequences which are the same when aligned for maximum correspondence over a specified comparison window. A “comparison window” refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci. U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.).

Alignment can also be performed by inspection and manual alignment. Typically, the polypeptides herein are at least 70%, generally at least 75%, optionally at least 80%, 85%, 90%, 95% or 99% or more identical to a reference polypeptide, e.g., an FOXO1 localization regulator encoded by a polynucleotide in Tables 1 and 2, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 95%, 99% or more identical to a reference nucleic acid, e.g., as measured by BLASTN (or CLUSTAL, or any other available alignment software) using default parameters. A “substantially identical” nucleic acid or amino acid sequence refers to a nucleic acid or amino acid sequence which comprises a sequence that has at least 90% sequence identity to a reference sequence using the programs described above (preferably BLAST) using standard parameters. The sequence identity is preferably at least 95%, more preferably at least 98%, and most preferably at least 99%.

As used herein, the term “insulin signaling related activity” encompasses any biochemical and physiological response caused or mediated by insulin signaling in regulating glucose homeostasis and regulating carbohydrate, lipid, and protein metabolism. Thus, it includes, e.g., insulin-stimulated receptor tyrosine kinase activity, insulin receptor substrate (IRS) phosphorylation or phosphoinositide (PI)-3 kinase activation, insulin-mediated activation or inactivation of transcription factors (e.g., FOXO1), and modulation of other gluconeogenesis and glycogenolytic activities. It also encompasses cell growth and proliferation in response to insulin signaling.

The term “modulate” with respect to a biological activity of a reference protein or its fragment refers to a change in the expression level or other biological activities of the protein. For example, modulation may cause an increase or a decrease in expression level of the reference protein, enzymatic modification (e.g., phosphorylation) of the protein, binding characteristics (e.g., binding to a target polynucleotide), or any other biological, functional, or immunological properties of the reference protein. The change in activity can arise from, for example, an increase or decrease in expression of one or more genes that encode the reference protein, the stability of an mRNA that encodes the protein, translation efficiency, or from a change in other biological activities of the reference protein. The change can also be due to the activity of another molecule that modulates the reference protein (e.g., a kinase which phosphorylates the reference protein).

Modulation of a reference protein can be up-regulation (i.e., activation or stimulation) or down-regulation (i.e. inhibition or suppression). The mode of action of a modulator of the reference protein can be direct, e.g., through binding to the protein or to genes encoding the protein, or indirect, e.g., through binding to and/or modifying (e.g., enzymatically) another molecule which otherwise modulates the reference protein.

The term “subject” includes mammals, especially humans. It also encompasses other non-human animals such as cows, horses, sheep, pigs, cats, dogs, mice, rats, rabbits, guinea pigs, monkeys. These subjects are all amenable for treatment with the insulin signaling-modulating compounds that can be identified in accordance with the present invention.

A “variant” of a reference molecule refers to a molecule substantially similar in structure and biological activity to either the entire reference molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

### III. SCREENING FOR MODULATORS OF FOXO1 AND INSULIN SIGNALING-GENERAL SCHEME

The FOXO1 localization regulators described above provide novel targets to screen for compounds that modulate FOXO1 activity and insulin signaling. Various biochemical and molecular biology techniques or assays well known in the art can be employed to practice the screening methods of the present invention. Such techniques are described in, e.g., Seethala et al., Handbook of Drug Screening, Marcel Dekker; 1st Ed. (2001); Janzen, High Throughput Screening: Methods and Protocols (Methods in Molecular Biology, 190), Humana Press; 1st Ed. (2002); Sambrook et al., Molecular Cloning: A Laboratory Manual, Cold Spring Harbor Press, N.Y., 3rd Ed. (2000); and Brent et al., Current Protocols in Molecular Biology, John Wiley & Sons, Inc. (ringbou ed., 2003). Further guidance to practice the screening methods of the present invention is provided below.

Typically, test agents or compounds are first assayed for their ability to modulate a biological activity of an FOXO1 localization regulator encoded by the cDNAs shown in Tables 1 and 2 (“the first assay step”). Modulating compounds thus identified are then subject to further screening for ability to modulate insulin signaling related activities, typically in the presence of the FOXO1 localization regulator (“the second testing step”). Depending on the FOXO1 localization regulator employed in the method, modulation of different biological activities of the FOXO1 localization regulator can be assayed in the first step. For example, the test agents can be screened for ability to modulate a known biochemical or enzymatic function of the FOXO1 localization regulator. The test agents can be assayed for activity to modulate expression or cellular level of the FOXO1 localization regulator, e.g., its transcription or translation. The test agents can also be screened for a specific binding activity to the FOXO1 localization regulator.

In some preferred embodiments, the FOXO1 localization regulator employed in the screening methods is an enzyme (e.g., a kinase or a phosphatase). In these methods, the biological activity monitored in the first screening step is the specific enzymatic activity of the FOXO1 localization regulator. The substrate to be used in the screening can be a molecule known to be enzymatically modified by the enzyme (e.g., a kinase), or a molecule that can be easily identified from candidate substrates for a given class of enzymes. For example, many kinase substrates are available in the art. See, e.g., www.emdbiosciences.com; and www.proteinkinase.de. In addition, a suitable substrate of a kinase can be screened for in high throughput format. For example, substrates of a kinase may be identified using the Kinase-Glo® luminescent kinase assay (Promega) or other kinase substrate screening kits (e.g., kits developed by Cell Signaling Technology, Beverly, Mass.).

The test agents can be screened for ability to either up-regulate or down-regulate a biological activity of the FOXO1 localization regulator in the first assay step. Once test agents that modulate the FOXO1 localization regulator are identified, they are typically further tested for ability to modulate insulin signaling activities, e.g., FOXO1 localization or tumor suppressing activities. This further testing step is often needed to confirm that their modulatory effect on the FOXO1 localization regulator would indeed lead to modulation of insulin signaling related activities (e.g., gluconeogenesis or tumorigenesis). For example, a test agent which inhibits a biological activity of an FOXO1 localization regulator may be further tested in order to confirm that such modulation can result in enhanced or reduced expression of FOXO1 and gluconeogenesis. Similarly, a test agent which stimulates a biological activity of an FOXO1 localization regulator that is a tumor suppressor gene can be further tested to confirm that it can lead to suppression of tumorigenesis.

In some embodiments, modulating compounds identified in the first screening step are examined in the second step to identify compounds that specifically inhibit FOXO1 localization. In some other embodiments, they are screened to identify compounds that enhance FOXO1 localization. In some of these applications, compounds that have been identified to modulate FOXO1 localization in the screening system are also examined for their impact on FOXO1 localization in a host that does not express FOXOlA. This step could confirm the compounds regulate FOXO1 localization in an FOXO1A-dependent manner.

In both the first assaying step and the second testing step, either an intact FOXO1 localization regulator, or a fragment thereof, may be employed. Analogs or functional derivatives of the FOXO1 localization regulator could also be used in the screening. The fragments or analogs that can be employed in these assays usually retain one or more of the biological activities of the FOXO1 localization regulator (e.g., kinase activity if the FOXO1 localization regulator employed in the first assaying step is a kinase). Fusion proteins containing such fragments or analogs can also be used for the screening of test agents. Functional derivatives of an FOXO1 localization regulator usually have amino acid deletions and/or insertions and/or substitutions while maintaining one or more of the bioactivities and therefore can also be used in practicing the screening methods of the present invention. A functional derivative can be prepared from an FOXO1 localization regulator by proteolytic cleavage followed by conventional purification procedures known to those skilled in the art. Alternatively, the functional derivative can be produced by recombinant DNA technology by expressing only fragments of an FOXO1 localization regulator that retain one or more of their bioactivities.

### IV. TEST COMPOUNDS

Test agents or compounds that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins. The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecule organic compounds, e.g., chemical compounds with a molecular weight of not more than about 1,000 or 500. Preferably, high throughput assays are adapted and used to screen such small molecules. In some methods, combinatorial libraries of small molecule test agents as described above can be readily employed to screen for small molecule compound modulators of insulin signaling. A number of assays are available for such screening, e.g., as described in Schultz (1998) Bioorg Med Chem Lett 8:2409-2414; Weller (1997) Mol. Divers. 3:61-70; Fernandes (1998) Curr Opin Chem Biol 2:597-603; and Sittampalam (1997) Curr Opin Chem Biol 1:384-91.

Libraries of test agents to be screened with the claimed methods can also be generated based on structural studies of the FOXO1 localization regulators discussed above or their fragments. Such structural studies allow the identification of test agents that are more likely to bind to the FOXO1 localization regulators. The three-dimensional structures of the FOXO1 localization regulators can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of the FOXO1 localization regulators' structures provides another means for designing test agents to screen for modulators of insulin signaling. Methods of molecular modeling have been described in the literature, e.g., U.S. Pat. No. 5,612,894 entitled “System and method for molecular modeling utilizing a sensitivity factor,” and U.S. Pat. No. 5,583,973 entitled “Molecular modeling method and system”. In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wuthrich (Wiley-Interscience, New York 1986).

Modulators of the present invention also include antibodies that specifically bind to an FOXO1 localization regulator in Tables 1 and 2. Such antibodies can be monoclonal or polyclonal. Such antibodies can be generated using methods well known in the art. For example, the production of non-human monoclonal antibodies, e.g., murine or rat, can be accomplished by, for example, immunizing the animal with an FOXO1 localization regulator or its fragment (See Harlow & Lane, Using Antibodies, A Laboratory Manual, Cold Spring Harbor Laboratory Press, Cold Spring Harbor N.Y., 1998). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to an FOXO1 localization regulator.

Human antibodies against an FOXO1 localization regulator can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using an FOXO1 localization regulator or its fragment.

### V. SCREENING FOR MODULATORS OF FOXO1 LOCALIZATION REGULATORS

Typically, test agents are first screened for ability to modulate a biological activity of an FOXO1 localization regulator listed in Tables 1 and 2. A number of assay systems can be employed in this screening step. The screening can utilize an in vitro assay system or a cell-based assay system. The biological activities of an FOXO1 localization regulator to be monitored in this screening step include its specific binding to the test agents, its expression or cellular level, and other biochemical or enzymatic activities of the FOXO1 localization regulator.

1. Modulating Binding Activities of FOXO1 Localization Regulators

In some methods, binding of a test agent to an FOXO1 localization regulator is determined in the first screening step. Binding of test agents to an FOXO1 localization regulator can be assayed by a number of methods including e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The test agent can be identified by detecting a direct binding to the FOXO1 localization regulator, e.g., co-immunoprecipitation with the FOXO1 localization regulator by an antibody directed to the FOXO1 localization regulator. The test agent can also be identified by detecting a signal that indicates that the agent binds to the FOXO1 localization regulator, e.g., fluorescence quenching or FRET.

Competition assays provide a suitable format for identifying test agents that specifically bind to an FOXO1 localization regulator. In such formats, test agents are screened in competition with a compound already known to bind to the FOXO1 localization regulator. The known binding compound can be a synthetic compound. It can also be an antibody, which specifically recognizes the FOXO1 localization regulator, e.g., a monoclonal antibody directed against the FOXO1 localization regulator. If the test agent inhibits binding of the compound known to bind the FOXO1 localization regulator, then the test agent also binds the FOXO1 localization regulator.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253, 1983); solid phase direct biotin-avidin EIA (see Kirkland et al., J. Immunol. 137:3614-3619, 1986); solid phase direct labeled assay, solid phase direct labeled sandwich assay (see, Harlow and Lane, “Antibodies, A Laboratory Manual,” Cold Spring Harbor Press, 3^{rd} ed., 2000); solid phase direct label RIA using ¹²⁵I label (see Morel et al., Mol. Immunol. 25(1):7-15, 1988); solid phase direct biotin-avidin EIA (Cheung et al., Virology 176:546-552, 1990); and direct labeled RIA (Moldenhauer et al., Scand. J. Immunol. 32:77-82, 1990). Typically, such an assay involves the use of purified polypeptide bound to a solid surface or cells bearing either of these, an unlabeled test agent and a labeled reference compound. Competitive inhibition is measured by determining the amount of label bound to the solid surface or cells in the presence of the test agent. Usually the test agent is present in excess. Modulating compounds identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize an FOXO1 localization regulator or its fragment onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the cellular regulator, the test agents are bound to the solid matrix and the FOXO1 localization regulator is then added.

Soluble assays include some of the combinatory libraries screening methods described above. Under the soluble assay formats, neither the test agents nor the FOXO1 localization regulator are bound to a solid support. Binding of an FOXO1 localization regulator or fragment thereof to a test agent can be determined by, e.g., changes in fluorescence of either the FOXO1 localization regulator or the test agents, or both. Fluorescence may be intrinsic or conferred by labeling either component with a fluorophor.

In some binding assays, either the FOXO1 localization regulator, the test agent, or a third molecule (e.g., an antibody against the FOXO1 localization regulator) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group. Similarly, the detectable group can be a substrate, cofactor, inhibitor or affinity ligand.

2. Modulating Other Activities of FOXO1 Localization Regulators

Binding of a test compound to an FOXO1 localization regulator provides an indication that the agent can be a modulator of the FOXO1 localization regulator. It also suggests that the agent may modulate FOXO1 activity and insulin signaling through, e.g., binding to and modulating the FOXO1 localization regulator. Thus, a test compound that binds to an FOXO1 localization regulator can be further tested for ability to modulate FOXO1 nuclear localization and/or other insulin signaling related activities such as gluconeogenesis (i.e., in the second testing step outlined above). Alternatively, a test agent that binds to an FOXO1 localization regulator can be further examined to determine whether it modulates another biological activity (e.g., an enzymatic activity) of the FOXO1 localization regulator. The existence, nature, and extent of such modulation can be tested by an activity assay as detailed below. Such an activity assay can confirm that the test agent binding to the FOXO1 localization regulator indeed modulates the FOXO1 localization regulator. More often, such activity assays can be used independently to identify test agents that modulate activities of an FOXO1 localization regulator (i.e., without first assaying their ability to bind to the FOXO1 localization regulator).

In general, the methods involve contacting a test agent with an FOXO1 localization regulator in the presence or absence of other molecules or reagents which are necessary to test a biological activity of the FOXO1 localization regulator (e.g., enzymatic activity if the FOXO1 localization regulator is an enzyme), and determining an alteration in the biological activity of the FOXO1 localization regulator. Preferably, FOXO1 localization regulators that are kinases or phosphatases are employed in the screening methods. Examples of kinases include Pik3r2, Dyrk3 and SGK. FOXO1 localization regulators which are protein tyrosine phosphatases include, e.g., PTP-MEG2 and PTPNl 8. In addition to these kinases and phosphatases, other enzymes shown in Tables 1 and 2 can also be readily employed to screen for modulators of their enzymatic activities. Examples include Tdg and Mat1a.

Many of these preferred FOXO1 localization regulators are well known and characterized in the art, e.g., phosphatases (e.g., PTP-MEG2 in Table 1) or kinases (e.g., SGK). Methods for assaying the enzymatic activities of these FOXO1 localization regulators are all routinely practiced in the art. For example, phosphatase activity of PTP-MEG2 can be assayed as described in, e.g., Kamatkar et al., J. Biol. Chem., 271:26755-26761, 1996 and Gu et al., J. Biol. Chem., 271:27751-27759, 1996. Similarly, kinase activity of SGK can be monitored using in vitro kinase assay as described in Brunet et al., Mol Cell Biol. 21:952-965, 2001. Activities of the other enzymes in Tables 1 and 2 can also be examined using assays that are known in the art.

In addition to assays for screening agents that modulate enzymatic or other biological activities of an FOXO1 localization regulator, the activity assays also encompass in vitro screening and in vivo screening for alterations in expression level of the FOXO1 localization regulator. Modulation of expression of an FOXO1 localization regulator can be examined in a cell-based system by transient or stable transfection of an expression vector into cultured cell lines. Test compounds can be screened for activity in altering expression level of a gene encoding the FOXO1 localization regulator in a cell, e.g., its mRNA level or protein level. These can be performed using methods well known and routinely practiced in the art, e.g., Samrbook et al., supra; and Brent et al., supra. More typically, test compounds are assayed for ability to modulate expression of a reporter gene (e.g., luciferase gene) under the control of a transcription regulatory element (e.g., promoter sequence) of an FOXO1 localization regulator. Genes encoding the FOXO1 localization regulators shown in Tables 1 and 2 have been characterized in the art. Their transcription regulatory elements such as promoter sequences have all been delineated.

Assay vector bearing the transcription regulatory element that is operably linked to the reporter gene can be transfected into any mammalian cell line for assays of promoter activity. Reporter genes typically encode polypeptides with an easily assayable activity (e.g., enzymatic activity) that is naturally absent from the host cell. Typical reporter polypeptides for eukaryotic promoters include, e.g., chloramphenicol acetyltransferase (CAT), firefly or Renilla luciferase, beta-galactosidase, beta-glucuronidase, alkaline phosphatase, and green fluorescent protein (GFP). Vectors expressing a reporter gene under the control of a transcription regulatory element of an FOXO1 localization regulator can be prepared using only routinely practiced techniques and methods of molecular biology (see, e.g., e.g., Samrbook et al., supra; Brent et al., supra). In addition to a reporter gene, the vector can also comprise elements necessary for propagation or maintenance in the host cell, and elements such as polyadenylation sequences and transcriptional terminators. Exemplary assay vectors include pGL3 series of vectors (Promega, Madison, Wis.; U.S. Pat. No. 5,670,356), which include a polylinker sequence 5′ of a luciferase gene. General methods of cell culture, transfection, and reporter gene assay have been described in the art, e.g., Samrbook et al., supra; and Transfection Guide, Promega Corporation, Madison, Wis. (1998). Any readily transfectable mammalian cell line may be used to assay expression of the reporter gene from the vector, e.g., HCT116, HEK 293, MCF-7, and HepG2 cells.

Modulation of expression of an FOXO1 localization regulator may also be detected by directly measuring the amount of RNA transcribed from a reporter gene under the control of a transcriptional regulatory element of the FOXO1 localization regulator. In these embodiments, the reporter gene may be any transcribable nucleic acid of known sequence that is not otherwise expressed by the host cell. RNA expressed from constructs containing an FOXO1 promoter or enhancer may be analyzed by techniques known in the art, e.g., reverse transcription and amplification of mRNA, isolation of total RNA or poly A⁺ RNA, northern blotting, dot blotting, in situ hybridization, RNase protection, primer extension, high density polynucleotide array technology and the like. These techniques are all well known and routinely practiced in the art.

### VI. SCREENING MODULATING COMPOUNDS FOR EFFECT ON INSULIN SIGNALING ACTIVITY

Once modulating compounds have been identified to bind to an FOXO1 localization regulator and/or to modulate the FOXO1 localization regulator (its expression level or other biological activities such as enzymatic activities), it can be further tested for activities in modulating FOXO1 activity or other insulin signaling related activities (e.g., gluconeogenesis). These include examining the modulating compounds for ability to regulate FOXO1 subcellular localization or regulate FOXO1-mediated target gene expression. The screening can also monitor other cellular activities regulated by FOXO1 and insulin signaling. Typically, this screening step is performed in the presence of the FOXO1 localization regulator on which the modulating compounds act. Preferably, this screening step is performed in vivo using cells that endogenously express the FOXO1 localization regulator. As a control, effect of the modulating compounds on insulin signaling-related activities in a cell that does not express the FOXO1 localization regulator can also be examined. When the screening methods are directed to identifying compounds that inhibit or enhance FOXO1 nuclear localization, FOXO1 is also expressed in the cell. FOXOlA can be expressed either endogenously by the host cell or from a separate expression vector that has been introduced into the host cell.

In some methods, the identified modulating compounds are further tested for ability to modulate (e.g., inhibit) FOXO1 nuclear localization. Effect of the compounds on FOXO1 nuclear localization can be examined using methods well known in the art, e.g., using cells that express a labeled FOXO1 or a FOXO1 protein fused with another molecule which can be easily traced or imaged (e.g., by fluorescence imaging). As exemplified in the Example below, U2OS cells expressing a GFP-FOXO1 fusion protein can be readily employed to screen the modulating compounds for ability to modulate FOXO1 subcellular localization. Following treatment with the compounds, the cells are then fixed and stained. The localization of FOXO1 inside the cell is then examined by imaging the cells with a fluorescence microscope.

In some other methods, the modulating compounds are screened for activity in modulating expression of a target gene that is regulated by FOXO1. To monitor activity of the modulating compounds on FOXO1-regulated expression of a target gene (e.g., G6Pase), typically a vector bearing a transcription regulatory element of the target gene that is operably linked to a reporter gene (e.g., a luciferase gene) is employed. As described above, a reporter gene-expressing vector can be transfected into any mammalian cell line. Preferably, the host cell does not express the reporter gene endogenously. The FOXO1 localization regulator with which the modulating compounds are identified in the first screening step can be either expressed endogenously by the cell or expressed from a second expression vector.

In still some other methods, the identified modulating compounds are further screened for ability to modulate other FOXO1-mediated cellular activities. For example, it is known that activated FOXO1 can induce cell death in LNCaP cells (Nakamura N et al., Mol Cell Biol. 20:8969-82, 2000). The modulating compounds can be examined for activity in modulating FOXO1-induced apoptosis with a cell viability assay. LNCaP cells can be put into contact with FOXO1 (see, e.g., Nakamura et al., Mol Cell Biol. 20:8969-82, 2000) in the presence of different modulating compounds described herein. Viability of the cells can then be examined, e.g., using the Cell Titer 96 aqueous nonradioactive cell proliferation assay (Promega). As control, LNCaP cells that have not been contacted with any of the modulating compounds or that have been contacted with a control compound can also be assessed in a viability assay. A further control that may be included in the screening is to test the compounds on viability of cells that are immune to FOXO1-induced cell death, e.g., 786-O cell (PTEN-null cell; Nakamura N et al., Mol Cell Biol. 20:8969-82, 2000). This control serves to confirm that any regulatory effect of a modulating compound on cell viability is mediated through FOXO1.

Other than identifying compounds that modulate FOXO1 localization or activities of other insulin signaling pathway members (e.g., expression of G6Pase), some of the screening methods are directed to identifying anti-tumor compounds. Some of the FOXO1 localization regulators may function as tumor suppressors which can inhibit insulin-mediated cellular proliferation by interfering with insulin signaling pathway. In these methods, test compounds are screened for ability to stimulate FOXO1 nuclear localization by either agonizing a stimulator of FOXO1 nuclear localization in Table 1 or antagonizing an inhibitor of FOXO1 nuclear localization in Table 2. For example, test compounds can be first screened to identify compounds which up-regulate expression or other biological activities (e.g., an enzymatic activity) of a stimulator of FOXO1 nuclear localization. Optionally, the identified modulating compounds are examined to confirm their activity in stimulating FOXO1 nuclear localization. The compounds can then be further tested in the second screening step for antitumor activities. Typically, the compounds are examined for ability to inhibit proliferation of a tumor cell in vitro. Preferably, this screening step is performed using cells that endogenously express the FOXO1 localization regulator. As a control, cytotoxicity of the modulating compounds on cells that do not express the cellular regulator can also be examined.

A variety of human tumor cell lines can be employed in this screening step, e.g., osteosarcoma cell line U2OS or glioblastoma cell line U373. Other tumor cell lines are available in the art, e.g., from American Type Culture Collection (Manassas, Va.). Antitumor cytotoxicity of the compounds can be monitored by measuring the IC₅₀ value (i.e., the concentration of a compound which causes 50% cell growth inhibition) of each of the modulating compounds. Preferably, an antitumor agent identified from this screening step will have an IC₅₀ value less than 1 μM on one or more of the tumor cell lines. More preferably, the IC₅₀ value of antitumor agents identified in accordance with the present invention is less than 250 nM. Some of the antitumor agents have an IC₅₀ value of less than 50 nM, less than 10 nM on at least one tumor cell line. Most preferably, the antitumor agents obtained from this screening step will have an IC₅₀ value that is less than 1 nM.

### VII. THERAPEUTIC APPLICATIONS

As demonstrated in the Examples below, insulin receptor activation can be regulated by modulating expression of a FOXO1 subcellular localization (e.g., PTP-MEG2). In addition, treating diabetic mice with agents (e.g., siRNAs) that down-regulate expression of PTP-MEG2 resulted in reduced insulin resistance and improved insulin sensitivity in the mice. Accordingly, by employing agents which modulate regulators of FOXO1 subcellular localization described herein, the present invention provides novel methods and compositions for modulating insulin signaling related activities, e.g., gluconeogenesis, and cell proliferation. These methods can be used either in vitro or in vivo to modulate (e.g., to increase) insulin sensitivity and/or to modulate glucose output by the liver cells. The methods also find application in treating a disease characterized by dysfunctional insulin signaling (e.g., resistance, inactivity or deficiency) and/or excessive glucose production. Modulation of insulin signaling related activities with the novel compounds of the present invention is also useful for preventing or modulating the development of such diseases or disorders in a subject.

A great number of diseases and conditions are amenable to treatment with methods and compositions of the present invention. Such diseases include, but are not limited to diabetes, hyperglycemia, obesity, and glycogen storage disease. For example, compounds that regulate FOXO1 nuclear localization (and hence gluconeogenesis) can also be employed to treat insulin resistance in type II diabetes. Type II diabetes is caused by faulty regulation of glucose metabolism and characterized by the initial development of insulin resistance, i.e. diminution in the ability of the cells to respond adequately to insulin. Elevated G6Pase activity is implicated in type II diabetes. Compounds which down-regulate FOXO1 nuclear localization are useful to treat or prevent the development of type II diabetes and hyperglycemia in a subject.

Obesity in humans and rodents is also commonly associated with insulin resistance. Before the development of diabetes, many obese patients develop a peripheral resistance to the actions of insulin. It was suggested that increased activities of key enzymes of pathways normally depressed by insulin contributes to insulin-resistance in obesity (Belfiore et al., Int J Obes 3:301-23, 1979). This failure of insulin to depress enzymes of catabolic pathways manifests itself in enhanced basal lipolysis in adipose tissue, increased amino acid release from muscle, and elevation in the activity of key gluconeogenic enzymes in the liver. Compounds which modulate (e.g., inhibit) gluconeogenesis can be employed to treat or prevent such disorders and conditions.

Similarly, novel compounds that modulate gluconeogenesis are also useful to treat glycogen storage diseases. Glycogen metabolism in the liver plays a major role in the homeostatic regulation of blood glucose levels. Glycogen storage diseases are known to be the result of genetic defects within the group of enzymes and transport proteins required by glycogen metabolism. Glycogen storage disease Type Iα (GSD, also known as yon Gierke disease) is defined as the deficiency of glucose-6-phosphatase which is normally present in liver, kidney, and intestine. Thus, compounds which modulate (e.g., enhance) FOXO1 localization can be employed to treat subjects with these diseases.

In some therapeutic applications of the present invention, therapeutic effects are monitored by measuring circulating glucose level in the subject before and/or after administering a compound that modulate insulin signaling pathway. Glucose level in the subject can be measured with methods well known in the art. For example, blood glucose levels can be measured very simply and quickly with many commercially available blood glucose testing kits.

Some of the therapeutic applications are directed to enhancing insulin signaling, e.g., treating insulin resistance. In these applications, typically an agent which down-regulates a stimulator of FOXO1 nuclear localization shown in Table 1 (e.g., PTP-MEG) is employed. Alternatively, an agent which up-regulates an inhibitor of FOXO1 nuclear localization shown in Table 2 can be employed in these applications. By way of example, a compound which down-regulates PTP-MEG cellular level or its enzymatic activity can be used to treat or ameliorate insulin resistance in a subject. Suitable compounds include agents that can be identified in accordance with the screening methods described above, small molecule compounds or antibodies (e.g., antagonist antibodies). They also include compounds which specifically inhibit expression or down-regulate cellular level of PTP-MEG.

In some therapeutic applications of the invention, nucleic acid agents which down-regulate PTP-MEG expression or cellular level can be employed. Such nucleic acid agents include, e.g., small interfering RNA (siRNA), short hairpin RNA (shRNA), anti-sense nucleic acid, microRNA (miRNA), or complementary DNA (cDNA). In some preferred embodiments, the therapeutic methods of the invention employ siRNA or shRNA agents that silence or deplete expression of PTP-MEG via RNA interference, as demonstrated in the Examples below. RNA interference (RNAi) is the process whereby the introduction of double stranded RNA (dsRNA) into a cell triggers degradation of homologous messenger RNA in the cytoplasm. See, e.g., Elbashir et al., Genes Dev. 15, 188-200, 2001; Elbashir et al., Nature 411: 494-498, 2001; and Hutvagner et al., Science 293:834-838, 2001. SiRNAs bind to a ribonuclease complex called RNA-induced silencing complex (RISC) that guides the small dsRNAs to its homologous mRNA target. Consequently, RISC cuts the mRNA approximately in the middle of the region paired with the antisense siRNA, after which the mRNA is further degraded.

Interference with the function and expression of endogenous genes by double-stranded RNA has been shown in various organisms such as C. elegans as described, e.g., in Fire et al., Nature 391:806-811, 1998; drosophilia as described, e.g., in Kennerdell et al., Cell 95:1017-1026, 1998; and mouse embryos as described, e.g., in Wianni et al., Nat. Cell Biol. 2:70-75, 2000. Other examples of making and using siRNAs and shRNAs for gene silencing in mammalian cells have been described in, e.g., Paddison et al., Genes Dev. 16:948-58, 2002; Paddison et al., Proc Natl Acad Sci USA 99:1443-1448, 2002; McCaffrey et al., Nature, 418:38-9, 2002; McManus et al., RNA, 8:842-50, 2002; Yu et al., Proc Natl Acad Sci USA 99:6047-52, 2002; Sui et al. Proc Natl Acad Sci USA, 8:5515-5520, 2002; Brummelkamp et al. Science, 296:550-553, 2002; Caplen et al., Proc Natl Acad Sci USA 98:9742-9747, 2001; and Elbashir et al., EMBO J. 20:6877-88, 2001.

SiRNAs are typically around 19-30 nucleotides in length, and preferably 21-23 nucleotides in length. They are double stranded, and may include short overhangs at each end. As described in the Examples below, siRNAs include chemically synthesized short RNA oligonucleotides (synthetic siRNAs). They also include short double-stranded RNAs produced in vivo using an RNAi expression vector that expresses a short hairpin RNA (shRNA) specific for a target gene (e.g., PTP-MEG). ShRNAs are single-stranded RNAs which contain a high-degree of secondary structure due to the presence of a stem-loop. As demonstrated in the Examples below, a short hairpin RNA can be produced by transferring a DNA vector molecule (e.g., adenovirus based vector) into a mammalian host cell where it is expressed in a short hairpin RNA. Upon transferring from the nucleus to the cytoplasm, the shRNA is chopped by the Dicer enzyme into siRNAs.

As illustrated in the Examples below, siRNAs targeting an FOXO1 localization regulator (e.g., PTP-MEG) can be prepared with methods well known in the art. They can be produced by chemical synthesis. They can also be recombinantly produced. Recombinant production of siRNAs can proceed by inserting a desired sequence in an expression vector. An RNA polymerase III (Pol III) promoter is then able to drive expression of both the sense and antisense strands separately, which then hybridize in vivo to make the siRNA. More often, as noted above, recombinant production of siRNAs involves using vectors and RNA Pol III to drive expression of single stranded shRNA which are then processed into siRNAs by the RNAi machinery.

Double stranded RNA like siRNAs can be introduced into a cell of interest (e.g., tumor cell) or a subject in a number of different ways. These include, e.g., microinjection, bombardment by particles covered by the dsRNA, soaking the cell in a solution of the dsRNA, electroporation of cell membranes in the presence of the dsRNA, liposome-mediated delivery of dsRNA and transfection mediated by chemicals such as calcium phosphate, viral infection, and transformation. In addition, dsRNA can also be supplied to a cell indirectly by introducing one or more vectors that encode both single strands of a dsRNA (or, in the case of a self-complementary RNA, the single self-complementary strand) into the cell. Preferably, the vector contains 5′ and 3′ regulatory elements that facilitate transcription of the coding sequence. Single stranded RNA is transcribed inside the cell, and, presumably, double stranded RNA forms and attenuates expression of the target gene.

All of the methods and techniques needed for performing RNAi are well known in the art. For example, WO 99/32619 (Fire et al., published 1 Jul. 1999) described how to supply a cell with dsRNA by introducing a vector from which it can be transcribed. Other teachings of RNAi are provided in, e.g., Reich et al., Mol. Vis. 9:210-6, 2003; Gonzalez-Alegre P et al., Ann Neurol. 53:781-7, 2003; Van De Wetering et al., EMBO Rep. 4:609-15, 2003; Miller and Grollman, DNA Repair (Amst) 2:759-63, 2003; Kawakami et al., Nat Cell Biol. 5:513-9, 2003; Abdelrahim et al., Mol. Pharmacol. 63:1373-81, 2003; Williams et al., J. Immunol. 170:5354-8, 2003; Daude et al., J Cell Sci. 116:2775-9, 2003; Jackson et al., Nat. Biotechnol. 21:635-7, 2003; Dillin, Proc Natl Acad Sci USA. 100:6289-91, 2003; Matta et al., Cancer Biol Ther. 2:206-10, 2003; Julien and Herr, EMBO J. 22:2360-9, 2003; Scherr et al., Cell Cycle. 2:251-7, 2003; Giri et al., J. Immunol. 170:5281-94, 2003; Liu and Erikson, Proc Natl Acad Sci USA. 100:5789-94, 2003; Chi et al., Proc Natl Acad Sci USA. 100:6343-6, 2003; Hall and Alexander, J. Virol. 77:6066-9, 2003.

Double stranded RNA can be introduced along with components that enhance RNA uptake by the cell, stabilize the annealed strands, or otherwise increase inhibition of the target gene. In the case of a cell culture or tissue explant, the cells are conveniently incubated in a solution containing the dsRNA or lipid-mediated transfection. For a subject (e.g., a non-human animal), the dsRNA can be conveniently introduced by injection or perfusion into a cavity or interstitial space of an organism, or systemically via oral, topical, parenteral (including subcutaneous, intramuscular and intravenous administration), vaginal, rectal, intranasal, ophthalmic, or intraperitoneal administration. In addition, the dsRNA can be administered via and implantable extended release device. Methods for oral introduction include direct mixing of RNA with food of the subject as well as engineered approaches in which a species that is used as food is engineered to express an RNA, then fed to the subject to be affected.

In addition to siRNAs and shRNAs, other nucleic acid agents targeting an FOXO1 localization regulator can also be employed in the methods of the present invention, e.g., microRNAs or antisense nucleic acids. MicroRNAs are short (about 21 nucleotides) RNAs, similar to siRNAs. Each microRNA originates with a gene that produces RNA that folds back on itself to form a short hairpin-like structure that is in turn processed into a microRNA. Antisense nucleic acids are DNA or RNA molecules that are complementary to at least a portion of a specific target mRNA molecule. In the cell, the single stranded antisense molecule hybridizes to that mRNA, forming a double stranded molecule. The cell does not translate an mRNA in this double-stranded form. Therefore, antisense nucleic acids interfere with the expression of mRNA into protein. Antisense methods have been used to inhibit the expression of many genes in vitro and in situ. See, e.g., Marcus-Sekura, Anal.Biochem., 172:289-295, 1988; Hambor et al., Proc. Natl. Acad. Sci. U.S.A. 85:4010-4014, 1988; Arima et al., Antisense Nucl. Acid Drug Dev. 8:319-327, 1998; Hou et al., Antisense Nucl. Acid Drug Dev. 8:295-308, 1998.

Other than silencing or depleting expression of a gene encoding a regulator of FOXO1 subcellular localization, the therapeutic applications of the invention can also employ agents that antagonize a biological activity of the regulator of FOXO1 subcellular localization protein (e.g., PTP-MEG). These include compounds that can be identified in accordance with the above described screen methods. Suitable agents that antagonizes a regulator of FOXO1 subcellular localization also include antagonist antibodies which specifically bind to the regulator of FOXO1 subcellular localization polypeptide and antagonize its biological activity. Monoclonal antibody-based reagents are among those most highly preferred in this regard. Such antagonist antibodies can be generated using methods well known and routinely practiced in the art, e.g., Monoclonal Antibodies—Production, Engineering, And Clinical Applications, Ritter et al., Eds., Cambridge University Press, Cambridge, UK, 1995; and Harlow and Lane, Antibodies, A Laboratory Manual, Cold Spring Harbor Press, 3^{rd} ed., 2000. Radiolabeled monoclonal antibodies for cancer therapy, in particular, are well known and are described in, for instance, Cancer Therapy With Radiolabelled Antibodies, D. M. Goldenberg, Ed., CRC Press, Boca Raton, Fla., 1995.

Compounds which modulate (e.g., down-regulate) expression or a biological activity of a regulator of FOXO1 subcellular localization (e.g., PTP-MEG) can be used in conjunction with other therapies. For example, subjects receiving surgery and radiation therapies can also be administered with a pharmaceutical composition of the present invention. In addition, chemotherapy, hormonal therapy and cryotherapy may also be combined with the therapeutic applications of the present invention to treat subjects suffering from cancers. The agents that modulate a regulator of FOXO1 subcellular localization can also be used in a subject to prevent tumor growth or treat cancer together with the administration of other therapeutic compounds for the treatment or prevention of these disorders. When an agent that modulates a regulator of FOXO1 subcellular localization is administered together with another anti-cancer agent, the two can be administered in either order or simultaneously. These therapeutic compounds may be chemotherapeutic agents, ablation or other therapeutic hormones, antineoplastic agents, monoclonal antibodies useful against cancers and angiogenesis inhibitors.

There are many anti-cancer drugs known in the art, e.g., as described in, e.g., Cancer Therapeutics: Experimental and Clinical Agents, Teicher (Ed.), Humana Press (1^{st} ed., 1997); and Goodman and Gilman's The Pharmacological Basis of Therapeutics, Hardman et al. (Eds.), McGraw-Hill Professional (10^{th} ed., 2001). Examples of suitable anti-cancer drugs include 5-fluorouracil, vinblastine sulfate, estramustine phosphate, suramin and strontium-89. Examples of suitable chemotherapeutic agents include Asparaginase, Bleomycin Sulfate, Cisplatin, Cytarabine, Fludarabine Phosphate, Mitomycin and Streptozocin. Hormones which may be used in combination with the present invention diethylstilbestrol (DES), leuprolide, flutamide, cyproterone acetate, ketoconazole and amino glutethimide.

### VIII. PHARMACEUTICAL COMPOSITIONS

The insulin signaling-modulating compounds of the present invention can be directly administered under sterile conditions to the subject to be treated. The modulators can be administered alone or as the active ingredient of a pharmaceutical composition. Therapeutic composition of the present invention can be combined with or used in association with other therapeutic agents. For example, a subject may be treated with a compound along with other conventional anti-diabetes drugs. Examples of such known anti-diabetes drugs include Actos (pioglitizone, Takeda, Eli Lilly), Avandia (rosiglitazone, Smithkline Beacham), Amaryl (glimepiride, Aventis), Glipizide Sulfonlyurea (Generic) or Glucotrol (Pfizer), Glucophage (metformin, Bristol Meyers Squibb), Glucovance (glyburide/metformin, Bristol Meyers Squibb), Glucotrol XL (glipizide extended release, Pfizer), Glyburide (Micronase; Upjohn, Glynase; Upjohn, Diabeta; Aventis), Glyset (miglitol, Pharmacia & Upjohn), Metaglip (glipizide+metformin; fixed combination tablet), Prandin (repaglinide, NOVO), Precose (acarbose, Bayer), Rezulin (troglitazone, Parke Davis), and Starlix (nateglinide, Novartis).

Further, some of the FOXO1 localization regulators disclosed in the present invention could be tumor suppressors. Compounds that modulate (e.g., stimulate) these tumor suppressors and inhibit tumorigenesis can be used to treat subjects with tumors. Examples of tumors that can be treated with methods and compositions of the present invention include various forms of tumors. The antitumor compounds of the present invention can be used alone or used in association with other therapeutic agents. For example, a subject may be treated concurrently with conventional chemotherapeutic agents, particularly those used for tumor and cancer treatment. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, vincristine, vinblastine, etoposide, trimetrexate, teniposide, cisplatin and diethylstilbestrol (DES). See, generally, The Merck Manual of Diagnosis and Therapy, 15th Ed., pp. 1206-1228, Berkow et al., eds., Rahay, N.J., 1987).

Pharmaceutical compositions of the present invention typically comprise at least one active ingredient together with one or more acceptable carriers thereof. Pharmaceutically carriers enhance or stabilize the composition, or to facilitate preparation of the composition. Pharmaceutically acceptable carriers are determined in part by the particular composition being administered (e.g., nucleic acid, protein, modulatory compounds or transduced cell), as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, or parenteral. For example, the antitumor compound can be complexed with carrier proteins such as ovalbumin or serum albumin prior to their administration in order to enhance stability or pharmacological properties.

There are a wide variety of suitable formulations of pharmaceutical compositions of the present invention (see, e.g., Remington: The Science and Practice of Pharmacy, Mack Publishing Co., 20^{th} ed., 2000). Without limitation, they include syrup, water, isotonic saline solution, 5% dextrose in water or buffered sodium or ammonium acetate solution, oils, glycerin, alcohols, flavoring agents, preservatives, coloring agents starches, sugars, diluents, granulating agents, lubricants, and binders, among others. The carrier may also include a sustained release material such as glyceryl monostearate or glyceryl distearate, alone or with a wax.

The pharmaceutical compositions can be prepared in various forms, such as granules, tablets, pills, suppositories, capsules, suspensions, salves, lotions and the like. The concentration of therapeutically active compound in the formulation may vary from about 0.1-100% by weight. Therapeutic formulations are prepared by any methods well known in the art of pharmacy. See, e.g., Gilman et al., eds., Goodman and Gilman's: The Pharmacological Bases of Therapeutics, 8th ed., Pergamon Press, 1990; Remington: The Science and Practice of Pharmacy, Mack Publishing Co., 20^{th} ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

The therapeutic formulations can be delivered by any effective means that can be used for treatment. Depending on the specific antitumor agent to be administered, the suitable means include oral, rectal, vaginal, nasal, pulmonary administration, or parenteral (including subcutaneous, intramuscular, intravenous and intradermal) infusion into the bloodstream. For parenteral administration, antitumor agents of the present invention may be formulated in a variety of ways. Aqueous solutions of the modulators may be encapsulated in polymeric beads, liposomes, nanoparticles or other injectable depot formulations known to those of skill in the art. Additionally, the compounds of the present invention may also be administered encapsulated in liposomes. The compositions, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The therapeutic formulations can conveniently be presented in unit dosage form and administered in a suitable therapeutic dose. A suitable therapeutic dose can be determined by any of the well known methods such as clinical studies on mammalian species to determine maximum tolerable dose and on normal human subjects to determine safe dosage. Except under certain circumstances when higher dosages may be required, the preferred dosage of an antitumor agent of the present invention usually lies within the range of from about 0.001 to about 1000 mg, more usually from about 0.01 to about 500 mg per day. The preferred dosage and mode of administration of an antitumor agent can vary for different subjects, depending upon factors that can be individually reviewed by the treating physician, such as the condition or conditions to be treated, the choice of composition to be administered, including the particular antitumor agent, the age, weight, and response of the individual subject, the severity of the subject's symptoms, and the chosen route of administration. As a general rule, the quantity of an antitumor agent administered is the smallest dosage which effectively and reliably prevents or minimizes the conditions of the subjects. Therefore, the above dosage ranges are intended to provide general guidance and support for the teachings herein, but are not intended to limit the scope of the invention.

### EXAMPLES

The following examples are offered to illustrate, but not to limit the present invention.

### Example 1

### Materials and Methods

High-throughput transfection and imaging: High-throughput (retro)transfections of 4800 human and mouse genes from the Mammalian Genome Collection (ref) were carried out essentially as described. Briefly, to pre-spotted 384-well black clear bottom plates containing 62.5 ng of plasmid DNA per well was added a 20 μL mixture of FuGENE 6 (Roche) and GFP-FOXO1 in pcDNA3 (20 ng/well) in DMEM medium (Invitrogen). After a 30-min incubation, approximately 2000 U2OS cells (ATCC) in 30 μL of DMEM supplemented with 16% FBS (Invitrogen) and 1 mM glutamine (Invitrogen) were added to each well. Cells were grown for 60 h at 37° C. in 5% CO₂. Cells were washed with PBS in an EMBLA plate washer (Molecular Devices) and fixed with 4% paraformaldehyde in PBS. Nuclei were stained with 4′,6-Diamidino-2-phenylindole (200 nM) in 0.3% TritonX-100/PBS for 30 min, and washed with PBS. Cells were imaged on an IC₁₀₀ (Beckman) automated inverted fluorescence microscope (Nikon TE300 with a Cohu video camera) with a 10×/0.5 objective (Nikon). Sixteen images were collected per well in two channels using filters appropriate for DAPI and GFP fluorophores. Image analysis was performed with CytoShop software (Beckman). After images were shade corrected and background subtracted, objects were extracted and single cells defined using geometric and total fluorescence parameters in the DAPI channel. Transfected cells were identified based on total GFP fluorescence and the fractional fluorescence in the nucleus (FLIN value) was determined for all GFP-positive cells. FLIN values were averaged for each well and wells with less than eight GFP-positive cells were excluded from further analysis. The number of standard deviations from the mean (σ value) for each gene was calculated as described in the main text.

Insulin receptorpulldown and anti-phosphotyrosine analysis: HEK-293A cells (Invitrogen) cultured in 10% FBS/DMEM/glutamine were transiently transfected with an empty vector, PTP-MEG2, or PTP-MEG2 (C515) using Effectene transfection reagent (Qiagen). After 2 days, cells were washed with PBS and serum starved in DMEM for 24 h. Cells were treated with bovine insulin (Sigma) at 10 nM concentration for 30 min. Cells were lysed in 1% triton X-100 in PBS with protease inhibitors (Roche), sodium fluoride (1 mM) and pervanadate (1 mM) and centrifuged at 14,000×g. Insulin receptor was immunoprecipitated from the supernatant with anti-insulin receptor β (Upstate Biotechnologies) overnight followed by binding to Protein G resin (Pierce). Resin was washed thoroughly and samples for electrophoresis were prepared by boiling in 3×SDS loading buffer. After SDS-PAGE electrophoresis proteins were transferred to 0.2 μm nitrocellulose membrane (Schleicher and Schuell) and probed with anti-phosphotyrosine antibody 4G10 (Upstate) or anti-insulin receptor β.

Transient overexpression and immunoblot analysis: HEK-293A cells were transfected with an empty vector control or PTP-MEG2 in 35 mm cell culture dishes. After 48 h cells were serum starved for 24 hours and treated with various concentrations of IGF-1 (Sigma). Lysates were prepared as described above, protein concentrations were determined, and equal quantities of protein were loaded in each lane for SDS-PAGE and transfer to nitrocellulose. Blots were probed with anti-InsR phosphor-Tyr1162/1163 (EMD Biosciences), anti-InsR β (Santa Cruz), or anti-β-actin (Sigma).

Small inhibitor RNA (siRNA) suppression analysis: HepG2 cells (ATCC) were cultured in MEM-α (Cellgro) supplemented with 10% FBS and glutamine at 37oC and 5% CO2. Cells were plated in 35 mm dishes and transfected with siRNA oligos mixed with Lipofectamine 2000 (Invitrogen) following the manufacturer's protocol. siRNA sequences were: MEG2 siRNA 5′-GCAUUUCCAGCUCGUUUGA-3′; mouse Trb3 siRNA 5′-CGAGUGAGAGAUGAGCCUG-3′ (ref); MEG2 smartpool: Dharmacon M-008832-00. After 48 h, cells were washed with PBS and serum starved in MEM-α/glutamine for 24 h. Cells were treated with insulin (3 nM) for 30 min and lysed as described above. Lysates were analyzed by western blot as described above. PTP-MEG2 levels were probed with a rabbit polyclonal antibody 1489B raised against the peptide sequence RPDMAPELTPEEE conjugated to keyhole limpet hemocyanin (Imgenex).

Short hairpin RNA (shRNA) mediated reduction of PTP-MEG2 expression: PTP-MEG2 RNAi adenovirus and a control RNAi adenovirus were used to infect glucose-6-phosphatase promoter-luciferase stable H4IIE cells (Alex ref) in 10% FBS/DMEM with glutamine. After 48 h, cells were serum starved in DMEM with glutamine for 24 h. Cells were treated with 25 μM dexamethasone (Sigma) and various concentrations of insulin for 24 h. Luciferase signals were read by the addition of BrightGlo reagent (Promega) and read on an Analyst plate reader (Molecular Devices).

Fasting and refeeding experiments: Wild-type C57/B16 adult male mice (6 per group) were fed ad libitum, fasted for 16 h, or fasted for 24 h and refed for 24 h. Total RNA was extracted from liver samples in Trizol (Invitrogen) using the RNase kit (Qiagen) with DNase treatment. cDNA was generated using the high capacity cDNA archive kit (Applied Biosystems). The real-time polymerase chain reaction (PCR) measurement was performed using Taqman probes labeled with carboxyfluorescein (FAM) dye for the relevant genes (PTPN9: probe Mm00451036_ml; G6Pase: probe Mm00839363_ml from Applied Biosystems) with a probe for mouse ribosomal protein 36B4 (VIC labeled) as an internal standard. The real time PCR was monitored on a Sequence Detection System 7900HT (Applied Biosystems) and reactions were run in triplicate.

### Example 2

### Identification of FOXO1 Modulators by cDNA Screening

To identify FOXO1 regulators, arrayed full-length cDNAs from the Mammalian Genome Collection were cotransfected with a GFP-FOXO1 reporter construct into U2OS cells using high-throughput methodology. The cDNAs were spotted in 384 well plates such that each well contained an individual cDNA with known identity. In a semi-automated process, cDNAs were incubated with a non-liposomal transfection reagent (Fugene, Roche Applied Science, Indianapolis, Ind.) and a GFP-FOXO1 reporter vector, pcDNA3-GFP-FKHR (Nakamura N et al., Mol Cell Biol. 20:8969-82, 2000). U2OS (human osteosarcoma cell line) cells were then introduced into each well to complete the transfection procedure. After 2 days of incubation at 37° C., 5% CO₂, cells were washed, fixed with paraformaldehyde, and stained with the nucleic acid binding dye DAPI. Cells were imaged using high throughput fluorescence microscopy on an IC100 system with data analysis performed using the CytoShop software platform (Beckman Coulter).

Two sets of images (one each for the DAPI and GFP fluorophores) were collected using automated fluorescence microscopy and were analyzed using CytoShop software. Image segmentation was performed in the images from the DAPI channel and objects that fell outside of experimentally determined parameters for total nuclear intensity and nuclear shape were excluded to ensure that only single cells were considered. The cytoplasm for each cell was defined as an annulus 30 pixels from the nuclear edge. The percentage of FOXO1 in the nucleus (FLIN) was determined for each transfected cell and averaged for each well. Cells that had extreme FLIN values (>95% or <5%) were excluded from the analysis since such values arose from segmentation or other artifacts (for example, GFP from the cytoplasm of a neighboring cell being included as part of a nontransfected cell). For each plate in the screen, the FLIN values of the middle 90% of the transfected wells were averaged, defining a plate mean and standard deviation, and the number of standard deviations from the mean (σ level) was calculated for each cDNA.

In U2OS cells, FOXO1 is localized in the cytoplasm in most cells but in both the cytoplasm and the nucleus in the rest. Cotransfection of GFP-FOXO1 with the lipid phosphatase PTEN, a known antagonist of PI3K/Akt signaling, results in a significant increase in the percentage of GFP-FOXO1 in the nucleus (50% to 64%, 6σ). For comparison, GFP-FOXO1 (T24A/S256A/S319A), a mutant in which the three Akt phosphorylation sites are mutated to alanine, has a FLIN value of 75% (11 σ). In contrast, overexpression of Akt1 results in GFP-FOXO1 cytoplasmic localization (FLIN 40%, −2.5 σ).

A number of genes identified in the screen have not been previously associated with FOXO1 signaling or have no known function. Several of them were confirmed to induce FOXO1 nuclear localization by >3.5 σ (Table 1), while other were found to decrease FOXO1 nuclear localization by <−2 σ (Table 2). In addition, among the genes identified in the screen, several are known to be in the PI3K/Akt pathway. Overexpression of the p85β subunit of PI3 kinase acts as a dominant negative, resulting in the strongest induction of GFP-FOXO1 nuclear localization (10 σ). Conversely, three 14-3-3 proteins (sigma, gamma, and zeta subtypes) were found to increase GFP-FOXO1 cytoplasmic localization, probably through direct binding to phosphorylated FOXO1.

### Example 3

### Characterization of PTP-MEG2 Function in Regulating Insulin Signaling

From the FOXO1 localization modulators identified from the cDNA screening, PTP-MEG2 (PTPN9) was chosen for follow-up study and validation. PTP-MEG2 is a nonreceptor tyrosine phosphatase with a 250-aa lipid binding domain that is homologous to Sec14p, a yeast protein with phosphatidylinositol (Ptdlns) transferase activity. To characterize the role of PTP-MEG2 in FOXO1 signaling, PTP-MEG2 and selected mutants were overexpressed in U2OS cells to study the effect on FOXO1 localization. The results indicate that while PTP-MEG2 overexpression stimulates FOXO1 localization in the nucleus, the catalytically inactive PTP-MEG2 (C515S) mutant did not have the same effect. In addition, expression of the PTP-MEG2 catalytic domain in the absence of the Sec14 homology domain resulted in a significant decrease in PTP-MEG2 activity. These results indicate that both catalytic activity and proper localization are required for PTP-MEG2 to increase FOXO1 nuclear localization.

To test if PTP-MEG2 overexpression inhibits the PI3K/Akt pathway, PTP-MEG2 was cotransfected with FOXO1 and GFP in HEK-293A cells, and transfected cells were isolated via fluorescence activated cell sorting. Western blot analysis showed that overexpression of PTP-MEG2 reduced FOXO1 phosphorylation at S256 suggesting that Akt activity is downregulated. Phosphorylation of endogenous Akt at S473, the PDK2 phosphorylation site, was also reduced, consistent with the observed downregulation of Akt activity, and suggesting that the PTP-MEG2 expression reduces PtdIns(3,4,5)P3 concentrations at the plasma membrane.

To determine the substrate of PTP-MEG2 activity responsible for the observed phenotype, PTP-MEG2 was overexpressed in HEK-293A cells which were serum-starved and treated with IGF-1. IP-MS analysis of PTP-MEG2 and control lysates using an anti-phosphotyrosine antibody revealed downregulation of IRS-1 and IGF1 receptor tyrosine phosphorylation when PTP-MEG2 is highly expressed. To verify that insulin/IGF1 receptors are dephosphorylated by PTP-MEG2 in cells, a vector control, PTP-MEG2, and PTP-MEG2(C515S) were transiently transfected into HEK-293A followed by serum starvation and insulin treatment. Immunoprecipitation of the insulin receptor β subunit followed by immunoblotting and probing with the anti-phosphotyrosine antibody 4G10 confirmed that PTP-MEG2 overexpression results in a reduction in insulin receptor phosphorylation. In addition, immunoblots of lysates from PTP-MEG2 transfected cells show that PTP-MEG2 expression reduces phosphorylation of Tyrl 162/1163 in the insulin receptor activation loop, sites whose phosphorylation is required for full kinase activity.

To assess whether endogenous PTP-MEG2 negatively regulates insulin receptor activity, we used RNA interference to reduce PTP-MEG2 expression in HepG2 cells. Transfection of a 21-mer duplex RNA oligonucleotide directed against human PTP-MEG2 as well as a mixture of four oligonucleotides (Smartpool) reduced PTP-MEG2 protein levels. Reduction of PTP-MEG2 levels resulted in potentiation of insulin receptor autophosphorylation in response to insulin treatment. Similar results were also seen in HEK-293A cells, suggesting that endogenous PTP-MEG2 downregulates insulin signaling in multiple insulin responsive cell lines.

### Example 3

### Decreasing Insulin Resistance by Antagonizing PTP-MEG2

Since reduction of PTP-MEG2 protein levels increases insulin's stimulation of insulin receptor phosphorylation, PTP-MEG2 loss of function would be expected to enhance insulin's suppression of gluconeogenic target genes in hepatic cell lines. To test this hypothesis, an adenovirus encoding a short hairpin RNA targeted against a rodent PTP-MEG2 sequence (Ad-MEG2 shRNA) was generated. Infection of H4IIE/G6Pase-luc hepatoma cells with Ad-MEG2 shRNA reduced the expression of PTP-MEG2 compared to control cells as assessed by immunoblot.

We also examined PTP-MEG2 expression levels in livers of fasted and refed mice. Adult male C57B1/6 mice (n=6 per group) were fasted for 16 h, or fasted for 24 h and refed for 24 h. Total RNA was prepared from liver and PTP-MEG2 and glucose-6-phosphatase (G6Pase) expression levels were measured using quantitative RT-PCR. G6Pase expression increased 349% during fasting (P<0.001) and decreased 21% (P<xxx) during refeeding, confirming that insulin signaling was downregulated during fasting and increased during refeeding. As expected, PEPCK expression increased 141% during fasting (P<0.001) and returned to normal after refeeding. PTP-MEG2 expression levels increased 33% in livers from fasted mice (P<0.001 by two-tailed Student's t test) but were 21% lower in refed mice (P<0.001) compare to fed mice. Thus PTP-MEG2 expression was 67% higher during insulin resistant conditions (fasting) compared to more insulin sensitive refeeding conditions.

To ascertain whether modulating the activity of PTP-MEG2 could reduce insulin resistance in diabetic (db/db) mice, we introduced adenovirus encoding a siRNA-hairpin directed against PTP-MEG2 to the liver of these animals. After fasting, mice were injected with insulin, and livers were harvested and analyzed by western blot analysis. When compared to liver extracts from non-specific RNAi treated mice, reduction of PTP-MEG2 levels resulted in an increase in insulin signaling as assessed by the phosphorylation status of insulin receptor and Akt. Correspondingly, the insulin-stimulated transcriptional repression of the gluconeogenic genes PGC-1 (and G6Pase was also markedly augmented in these animals. We next performed glucose tolerance tests (GTT) on db/db mice injected with adenovirus encoding MEG2 or control siRNAs. These studies reveal that attenuation of hepatic PTP-MEG2 expression in db/db mice leads to a significant reduction in both the amplitude and duration of elevated blood sugar levels. Finally, we measured blood glucose levels in the MEG2 and control siRNA infected db/db mice either under fasted conditions or an ad libitum feeding regimen. Reduction of PTP-MEG2 resulted in decrease in blood glucose levels during fasting, and lead to a significant reversal of hyperglycemia under ad lib feeding. Taken together, these data indicate that the antagonism of PTP-MEG2 activity can restore insulin sensitivity and glycemic regulation in diabetic mice.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, ATCC deposits, patents and patent applications cited herein are hereby expressly incorporated by reference in their entirety and for all purposes as if each is individually so denoted.

We claim:

1. A method of treating or ameliorating insulin resistance in a subject, comprising administering to the subject a pharmaceutical composition comprising an effective amount of a compound which down-regulates cellular level or enzymatic activity of PTP-MEG2, thereby treating insulin resistance in the subject.

2. The method of claim 1, wherein the subject is a human.

3. The method of claim 1, wherein the subject suffers from type II diabetes.

4. The method of claim 1, wherein the agent down-regulates cellular level of PTP-MEG2.

5. The method of claim 4, wherein the agent is a short interfering RNA (siRNA) that specifically targets PTP-MEG2.

6. The method of claim 5, wherein the siRNA agent is around 19-30 nucleotides in length.

7. The method of claim 5, wherein said siRNA agent is 21-23 nucleotides in length.

8. The method of claim 5, wherein said siRNA agent is chemically synthesized.

9. The method of claim 5, wherein said siRNA agent is produced in vivo with a PTP-MEG2 RNAi expression vector.

10. The method of claim 9, wherein the PTP-MEG2 RNAi expression vector is an adenovirus based vector.

11. The method of claim 4, wherein the agent is an anti-sense nucleic acid that specifically targets PTP-MEG2.

12. A method for identifying an agent that modulates insulin signaling, the method comprising:
(a) screening test compounds to identify one or more modulating compounds which modulate an FOXO1 localization regulator encoded by a polynucleotide selected from the members listed in Tables 1 and 2; and
(b) testing the modulating compounds for ability to modulate an insulin signaling related activity.

13. The method of claim 12, wherein the modulating compounds down-regulate the FOXO1 localization regulator.

14. The method of claim 12, wherein the modulating compounds up-regulate the FOXO1 localization regulator.

15. The method of claim 12, wherein the FOXO1 localization regulator is an enzyme, and the modulating compounds modulate an enzymatic activity of the FOXO1 localization regulator.

16. The method of claim 12, wherein the modulating compounds modulate expression of the FOXO1 localization regulator.

17. The method of claim 12, wherein the FOXO1 localization regulator is an inhibitor of FOXO1 nuclear localization shown in Table 2.

18. The method of claim 12, wherein the FOXO1 localization regulator is a stimulator of FOXO1 nuclear localization shown in Table 1.

19. The method of claim 18, wherein the FOXO1 localization regulator is PTP-MEG2.

20. The method of claim 19, wherein the modulating compounds inhibit the phosphatase activity of PTP-MEG2.

21. The method of claim 19, wherein the modulating compounds down-regulate cellular level of PTP-MEG2.

22. The method of claim 12, wherein (b) comprises testing the modulating compounds for ability to modulate subcellular localization of FOXO1.

23. The method of claim 12, wherein (b) comprises testing the modulating compounds for ability to modulate FOXO1-mediated expression of an insulin signaling pathway member.
